(19) United States
(12) Patent Application Publication
(10) Pub. No.: US 2025/0046392 A1
(43) Pub. Date: Feb. 6, 2025

(54) SYSTEM AND METHOD FOR AIDING DRUG DEVELOPMENT

(71) Applicant: Innoplexus AG, Eschborn (DE)

(72) Inventors: Vinay Chandil, Gwalior (IN); Om Sharma, Pimpri-Chinchwad (IN); Sonali Agarwal, Agra (IN)

(73) Assignee: Innoplexus AG, Eschborn (DE)

(21) Appl. No.: 18/362,199

(22) Filed: Jul. 31, 2023

(51) Int. Cl. G16B 15/30

(52) U.S. Cl. CPC G16B 15/30

(57) ABSTRACT

System (100) and method (200) for aiding drug development by determining a curable action value of a target protein are disclosed. The system (100) comprises a protein data extraction module (110), a protein data filtration module (120), and a final expression level calculator (150). The protein data extraction module (110) is configured for parsing and identifying information related to target proteins from a public database. The public database comprises data related to proteins. The protein data filtration module (120) is configured for filtering out irrelevant information from the identified information related to the target proteins. The final expression level calculator (150) is configured for calculating the final expression level of the identified relevant information. The curable action of the target protein is based upon the final expression level. The invention aids in drug development by determining the curable action of the protein in the disease based on biomedical data corpus.

### FIELD OF THE INVENTION

The present invention relates to drug development and more particularly to system and method for aiding drug development.

### BACKGROUND OF THE INVENTION

In drug discovery, the process of determining the role of a target protein in a disease condition is crucial for the development of a drug and therefore, curing the disease. During the process of drug development, determining the exact role and impact of the target protein is not only cumbersome but also tedious as it involves availability and access to laboratory and computational techniques in order to determine the exact expression of the target protein-high, low in healthy, control, etc.

Such information is important and plays a critical role to aid the development of the drug, be it an agonist or antagonist, or the like, to ascertain the impact and to cure the disease. Mechanism of Action (MoA) refers to a biochemical process through which a drug produces its effect. Understanding of the MoA for any drug, which describes the functional changes at molecular or cellular level, is a critical step in the drug discovery process. Determining the MoA of bioactive compounds is crucial as it helps in the determination of a better dose regimen, clinical safety and the best combination therapy that can reduce the likelihood of drug resistance.

A large corpus of data forming part of such information about the impacts of protein is available in the biomedical repositories in the form of textual data, but the data is unorganised and scattered. The vastness, and the scattered and unorganised nature of the data available makes it rather difficult, if not impossible, to ascertain definitively the impacts of protein on a related disease. Also, a lot of such data may not be useful for being just a cursory discussion.

Besides, in this digital age where data is created and made available at lightning speed, the corpus of data related to the impacts of proteins keeps on updating dynamically. It is desired to keep pace with the changing nature of this data during the expensive and tedious process of drug development to search for the literature evidence consisting of particular keywords and validate the sentences to tag the MOA of protein in disease.

Because of the dynamic nature of this data, which adds to its scattered and unorganised nature, it becomes rather impossible to come to the most updated and definitive relationship between a protein and a related disease as per the available data corpus.

Therefore, there is a need for systems and methods which can identify the exact updated relationship between a protein and a disease to aid drug discovery and development.

### SUMMARY OF THE INVENTION

An object of the invention is to provide systems and methods to determine a curable action value of a target protein for aiding drug discovery and development, which is less time consuming, less cumbersome and more efficient.

Another object of the invention is to aid in drug development by determining the role of protein in a disease and also predict the curable action of a target protein based on the public biomedical literature corpus.

Accordingly, in one aspect the invention provides a system determining a curable action value of a target protein for aiding in drug development. In accordance with an embodiment of the invention, the system comprises a protein data extraction module, a protein data filtration module, and a final expression level calculator. The protein data extraction module is configured for parsing and identifying information related to target proteins from a public database. The public database comprises data related to proteins. The protein data filtration module is configured for filtering out irrelevant information from the identified information related to the target protein. The final expression level calculator is configured for calculating the final expression level of the identified relevant information. The curable action of the target protein is based upon the final expression level.

In an embodiment, the protein data filtration module comprises a first-level logistic regression ML Classifier, a second-level logistic regression ML Classifier, and a third-level logistic regression ML Classifier. The first-level ML classifier is configured for classifying each of the relevant word strings into a first-level classification. The first-level classification is one of a conclusive word string and an inconclusive word string done based upon the nature of the relevant word string. The second-level ML classifier is configured for classifying each of the conclusive word strings into one of a high expression word string and a low expression word string. The second-level classification is done based upon the expression level of the protein. The third-level ML classifier is configured for classifying each of the conclusive word strings into one of an inhibit effect, an activate effect and an associate effect based upon the impact of the protein on the disease.

In an embodiment, the protein data extraction module comprises a string extraction module, a set of keywords, and a string filtering module. The string extraction module is configured for identifying strings of words in the public database related to the target protein. The identification is done by searching for the target protein and a related disease within the public database. The set of keywords includes a set of words. The string filtering module is configured for filtering relevant word strings from the identified strings of words. The filtering is performed by checking the presence of at least one word from the set of keywords in the identified string of words. The set of keywords consists of a combination of at least one word chosen from a group consisting of mutat, express, polymorph, regulat, level, associat, inhibit, activat, high, low, less, up, and down.

In an embodiment, the system comprises a probability filtering module. The probability filtering module is configured to assign a predefined probability value to the conclusive word strings, and identify high probability word strings from the conclusive word strings by comparing an assigned probability value with a predefined threshold probability value. The predefined threshold probability value is input by a user of the system.

In an embodiment, the system comprises a grouping module. The grouping module is configured to aggregate the identified high probability word strings into groups based upon the disease to which the target protein in the high probability word strings pertains. The aggregated high probability word strings is the identified relevant information. The final expression level calculator calculates the final expression level from the identified relevant information by comparing the probability values of the word strings.

In various embodiments, the public data is in the form of a dynamic digital data, a dynamic physical data, a combination of either, and the like. The system includes a Natural Language Processing (NLP) module. For example, the protein data extraction module uses the NLP module for parsing and identifying information related to the target proteins.

In another aspect, the invention provides a method for determining a curable action value of a target protein for aiding in drug development. In accordance with an embodiment of the invention, the method comprises the steps of: parsing and identifying information related to the target protein from a public database, the public database comprising data related to proteins; filtering out irrelevant information from the identified information related to the target proteins; and calculating a final expression level of the identified relevant information, the curable action of the target protein being based upon the final expression level.

In an embodiment, the step of parsing and identifying comprises the steps of identifying strings of words in the public database related to the target protein, and filtering relevant word strings from the identified strings of words. The identifying is done by searching for the target protein and a related disease within the public database using a string extraction module. The filtering is performed by checking the presence of at least one word from a set of keywords in the identified string of words using a string filtering module.

In an embodiment, the step of filtering comprises the steps of classifying each of the relevant word strings into a first-level classification based upon the nature of the relevant word string into one of a conclusive word string and an inconclusive word string; classifying each of the conclusive word strings into a second-level classification based upon the expression level of the protein into one of a high expression word string and a low expression word string; and classifying each of the conclusive word strings into a third-level classification based upon the impact of the protein on the related disease into one of an activate effect and an associate effect.

In an embodiment, the method comprises the steps of assigning a predefined probability value to the conclusive word strings and identifying high probability word strings from the conclusive word strings by comparing an assigned probability value with a predefined threshold probability value. The predefined threshold probability value is chosen and input by a user.

In an embodiment, the method comprises the steps of aggregating the identified high probability word strings into groups based upon the disease to which the target protein in the high probability word strings pertains. The aggregated high probability word strings is the identified relevant information. The step of calculating includes comparing the probability values of the word strings.

In an embodiment the step of calculating includes calculating the final expression level from the identified relevant information by comparing the probability values of the word strings.

In various embodiments, the steps of the method may be performed using a Natural Language Processing (NLP) module.

### BRIEF DESCRIPTION OF THE DRAWINGS

The method and system are illustrated in the accompanying drawings, throughout which like reference letters indicate corresponding parts in the various drawings. The invention herein will be better understood from the following description with reference to the drawings, in which:

FIG. 1 is a block diagram illustrating a system for aiding drug development, in accordance with an embodiment of the invention.

FIG. 2 is a flowchart illustrating a method for aiding drug development, in accordance with an embodiment of the invention.

FIG. 3 is a flowchart illustrating the method for aiding drug development, in accordance with an embodiment of the invention.

FIG. 4 is a block diagram illustrating an exemplary embodiment of the system for aiding drug development, in accordance with an embodiment of the invention.

FIG. 5 is a table illustrating a sample result set from a first-level logistic regression ML Classifier of the system, in accordance with an embodiment of the invention.

FIG. 6 is a table illustrating a sample result set from a second-level logistic regression ML Classifier of the system, in accordance with an embodiment of the invention.

FIG. 6A is a table illustrating a sample result set in descending order of the probability as assigned by the second-level logistic regression ML Classifier, in accordance with an embodiment of the invention.

FIG. 7 is a table illustrating a sample result set from a third-level logistic regression ML Classifier, in accordance with an embodiment of the invention.

FIG. 7A is a table illustrating a sample result set, wherein the strings of words are arranged with corresponding probability as assigned by the third-level logistic regression ML Classifier, in accordance with an embodiment of the invention.

FIG. 8 is a table illustrating an aggregated result set, in accordance with an embodiment of the invention.

FIG. 9 is a table illustrating an example result in the form of a matrix for a target protein, in accordance with an embodiment of the invention.

### DETAILED DESCRIPTION

In the following detailed description of the invention, numerous specific details are set forth in order to provide a thorough understanding of the invention. However, it will be obvious to a person skilled in the art that the invention may be practiced with or without these specific details. In other instances, well known methods, procedures and components have not been described in details so as not to unnecessarily obscure aspects of the invention.

Furthermore, it will be clear that the invention is not limited to these implementations only. Numerous modifications, changes, variations, substitutions and equivalents will be apparent to those skilled in the art, without parting from the scope of the invention. The accompanying drawings are used to help easily understand various technical features and it should be understood that the implementations presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

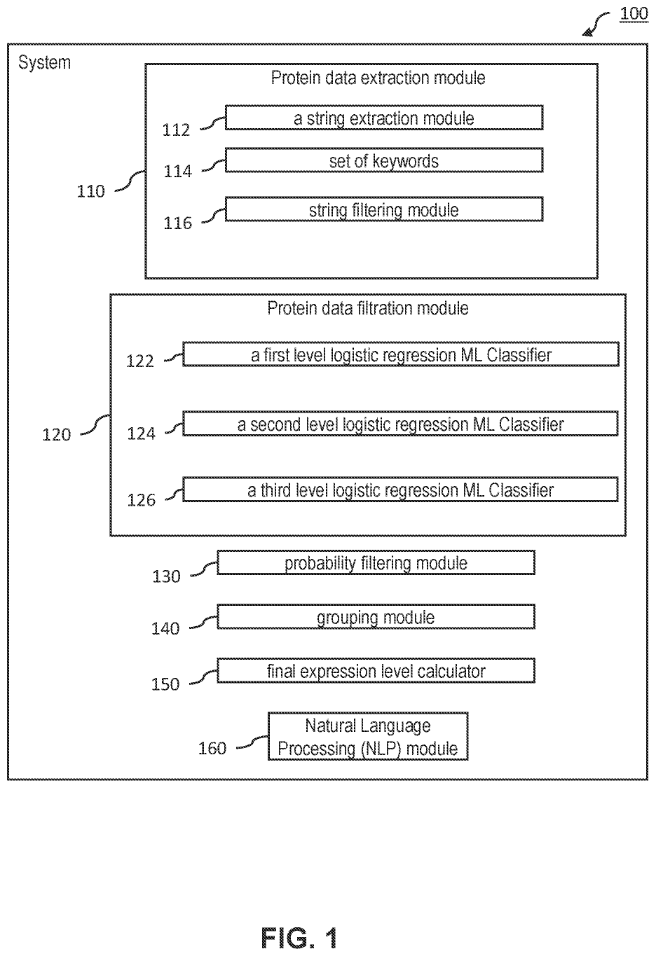

FIG. 1 is a block diagram illustrating a system (100) for determining a curable action value of a target protein for aiding in drug development, in accordance with an embodiment of the invention. The system (100) comprises a protein data extraction module (110), a protein data filtration module (120), and a final expression level calculator (150). The protein data extraction module (110) is configured for parsing and identifying information related to target proteins from a public database. The protein data filtration module (120) is configured for filtering out irrelevant information from the identified information related to the target proteins. The final expression level calculator (150) is configured for calculating the final expression level of the protein from the identified relevant information. The curable action of the target protein is based upon the final expression level.

In various embodiments, the public database comprises information and data related to proteins and the effect of protein on a related disease. In an embodiment, the public database may include biomedical textual data like publications, news, thesis and congresses, and may be a free data base available online on a network such as the internet, LAN and the like, or in the form of a dynamic digital data on a physical storage medium, or a dynamic physical data, or a combination of these, and the like. In an embodiment, the public database may be a proprietary data based upon subscription or payment to which the system (100) has real time access.

In an embodiment, the system (100) includes a data tracking module to keep track of any updates in the public database. The system (100) may be configured to run and execute for the update information in the public database only automatically at predetermined intervals of time or by receiving an instruction from a user or operator of the system (100).

In an embodiment, the protein data extraction module (110) comprises a string extraction module (112), a set of keywords (114), and a string filtering module (116). The string extraction module (112) is configured for identifying strings of words in the public database related to the target protein by searching for the target protein and the related disease within the public database. In an embodiment, a user may only input a target protein and the string extraction module (112) may only search for the target protein. The related disease may then be identified based upon the strings of words so received. The strings of words may be in the form of a sentence and may be referred to as sentence extraction. The set of keywords (114) includes a set of words. The string filtering module (116) is configured for filtering relevant word strings from the identified strings of words by checking the presence of at least one word from the set of keywords in the identified string of words. In an embodiment, the system (100) includes a c-occur entity module for including various synonyms of the target protein while searching for the string of words.

In various embodiments, the set of keywords (114) consists of at least one word chosen from a group consisting of mutat, express, polymorph, regulat, level, associat, inhibit, activat, high, low, less, up, and down. This may be referred to as ‘Bag of Words.’

For example, in an example embodiment, a user inputs into the system (100), a target (gene/protein) based on the requirement to determine the role of a target protein in a related disease-condition. It may be appreciated that the user may only input the target protein without identifying or inputting a related disease-condition. The system (100) searches for the presence of the protein and the disease-condition (if provided/input) for which the association is to be derived using the protein data extraction module (110). Strings of words such as sentences are mined from publications (public database) which include the disease condition and the target protein of interest in the text.

In various embodiments, the system (100) may include a Natural Language Processing (NLP) module (160). For example, the protein data extraction module (110) may use the NLP module (160) for parsing and identifying information related to the target proteins.

Continuing with reference to the example embodiment, the sentences retrieved from the publications are further narrowed down using the set of keywords (114), and the string filtering module (116). If at least one of the words/terms or a combination is present in the sentence, the sentence is selected for further processing as per system (100), else it is dropped. An example list of Bag of Words: “mutat”, “express”, “polymorph”, “regulat”, “level”, “associat”, ‘inhibit’, ‘activat’, ‘high’, ‘low’, ‘less’, ‘up’, ‘down’ and the like.

It may be apparent that this list is not exhaustive and may be updated and more words added to the bag of words as per the requirement and to fine tune the system (100). The filtering using the set of keywords (114) and the string filtering module (116) helps to reduce the amount of irrelevant sentences associated with the protein and the related disease and only retains the text with protein expression details.

In an embodiment, the protein data filtration module (120) comprises a first-level logistic regression ML Classifier (122), a second-level logistic regression ML Classifier (122), and a third-level logistic regression ML Classifier (122). The first-level ML classifier (122) is configured for classifying each of the relevant word strings into a first-level classification which is one of a conclusive word string and an inconclusive word string based upon the nature of the relevant word string. The second-level ML classifier (124) is configured for classifying each of the conclusive word strings into one of a high expression word string and a low expression word string based upon the expression level of the protein. The third-level ML classifier (126) is configured for classifying each of the conclusive word strings into one of an inhibit effect, an activate effect and an associate effect based upon the impact of the protein on the disease.

Continuing with reference to the example embodiment, the three independent machine learning algorithms models, the first-level logistic regression ML Classifier (122), the second-level logistic regression ML Classifier (122), and the third-level logistic regression ML Classifier (122) of the protein data filtration module (120) are self learning algorithms and are trained through iterations each performing a certain level of filtration and classification to refine and identify relevant information between the protein and disease.

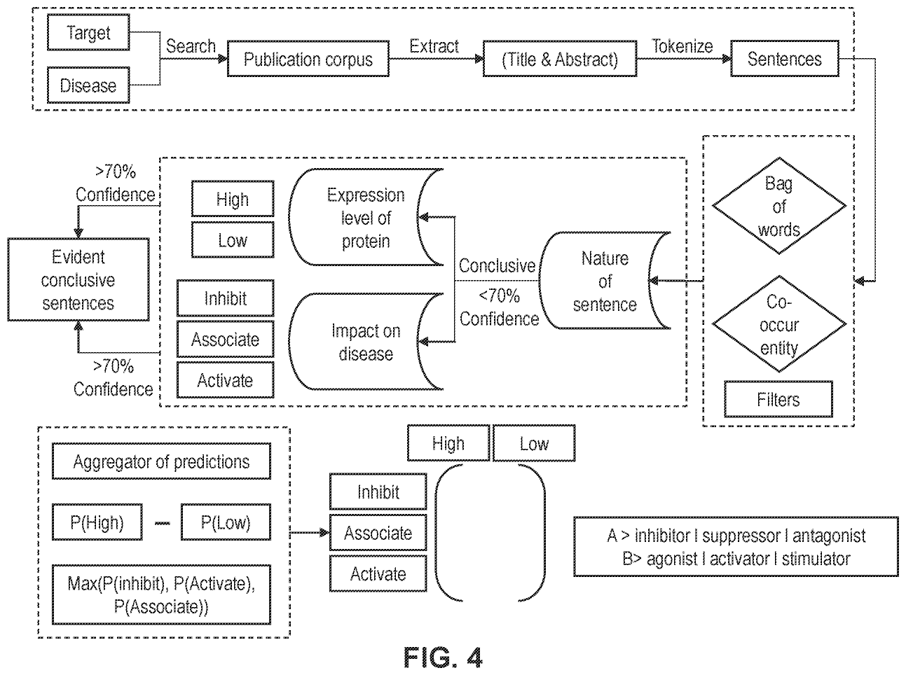

FIG. 4 is a block diagram illustrating an exemplary embodiment of the system (100) for aiding drug development, in accordance with an embodiment of the invention.

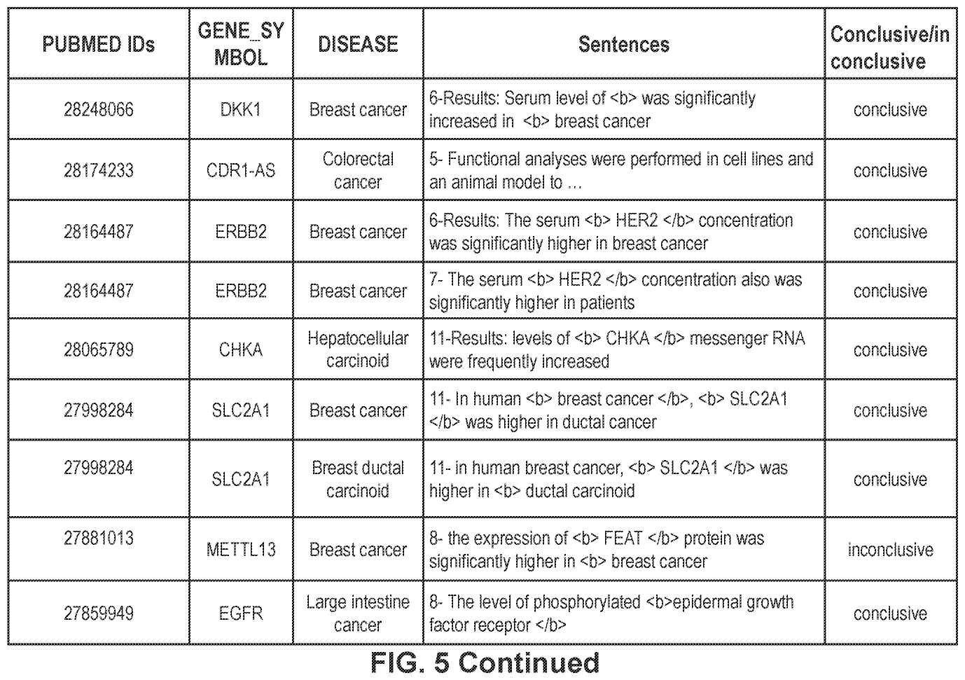

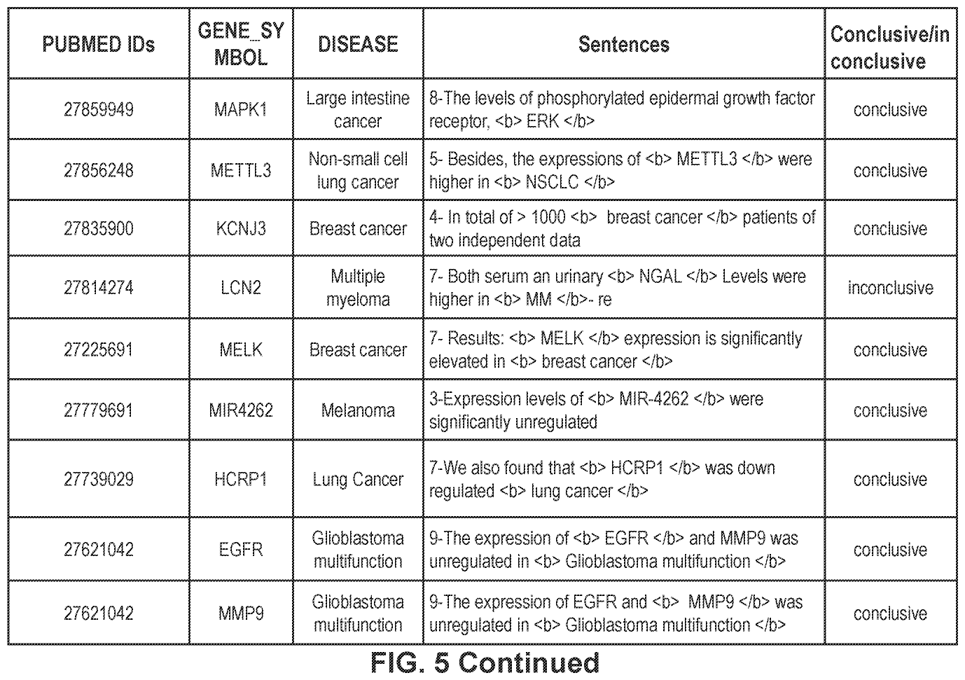

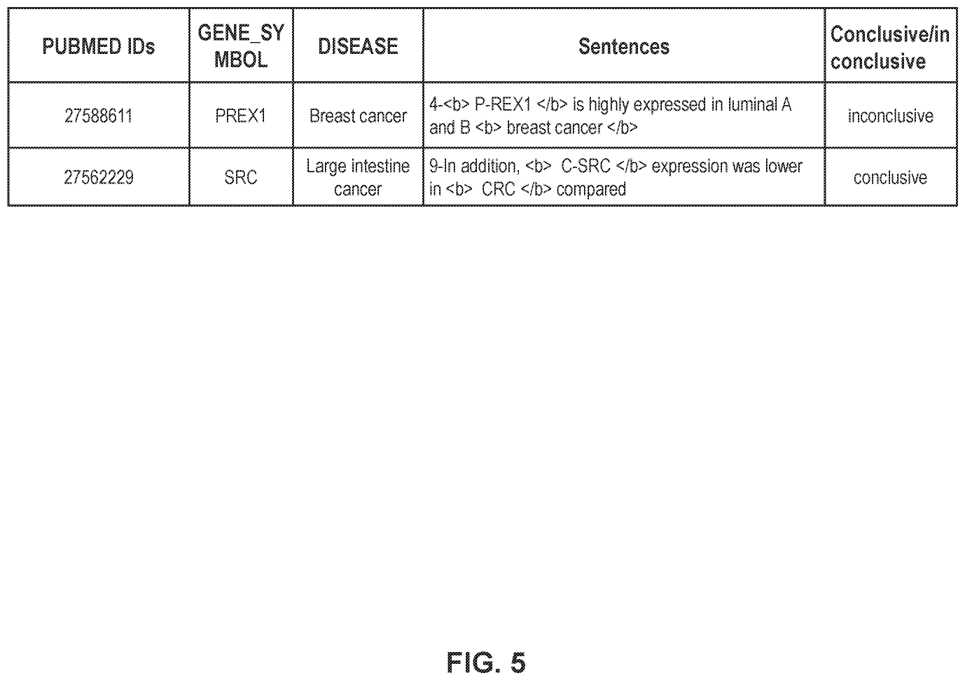

As an example, FIG. 5 is a table illustrating a sample result set from the first-level logistic regression ML Classifier (122), in accordance with an embodiment of the invention. The first-level logistic regression ML Classifier (122) is configured for classifying based upon the nature of the sentence. Many sentences in publications are often inconclusive or are just hypotheses. Segregating conclusive statements from inconclusive ones is important to rule out for further processing as they should not be used as evidence of any association. Two tags Conclusive and Inconclusive are used to tag the Sentences. Inconclusive Sentences are dropped out.

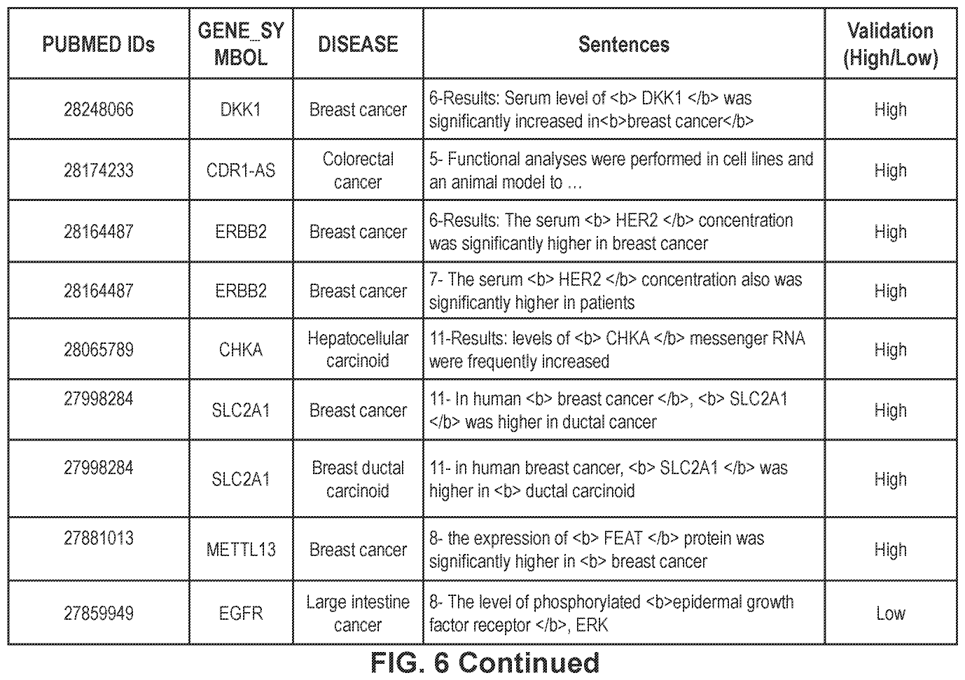

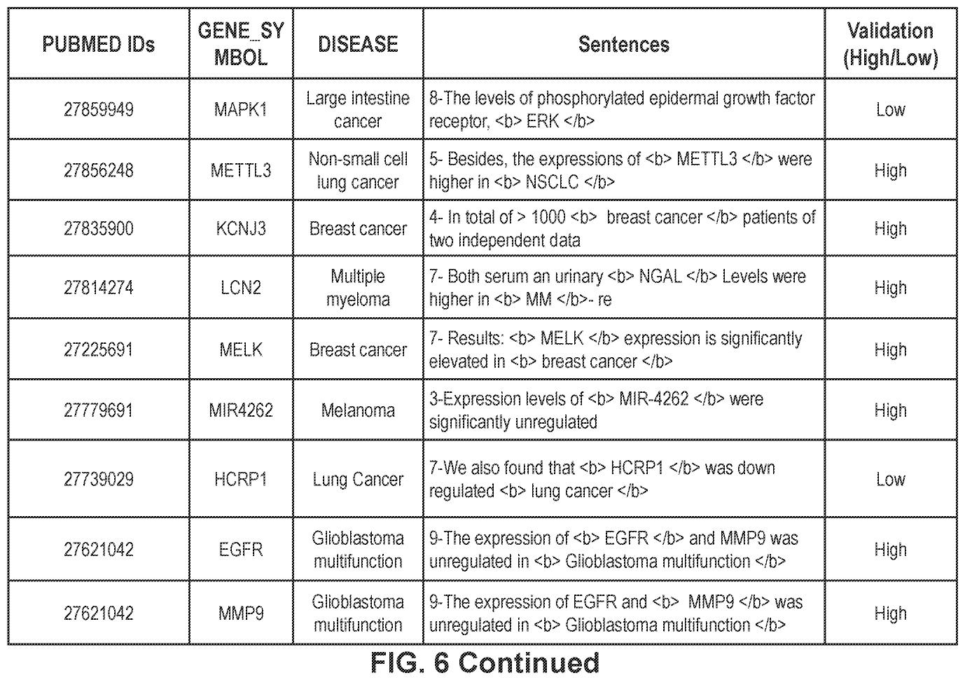

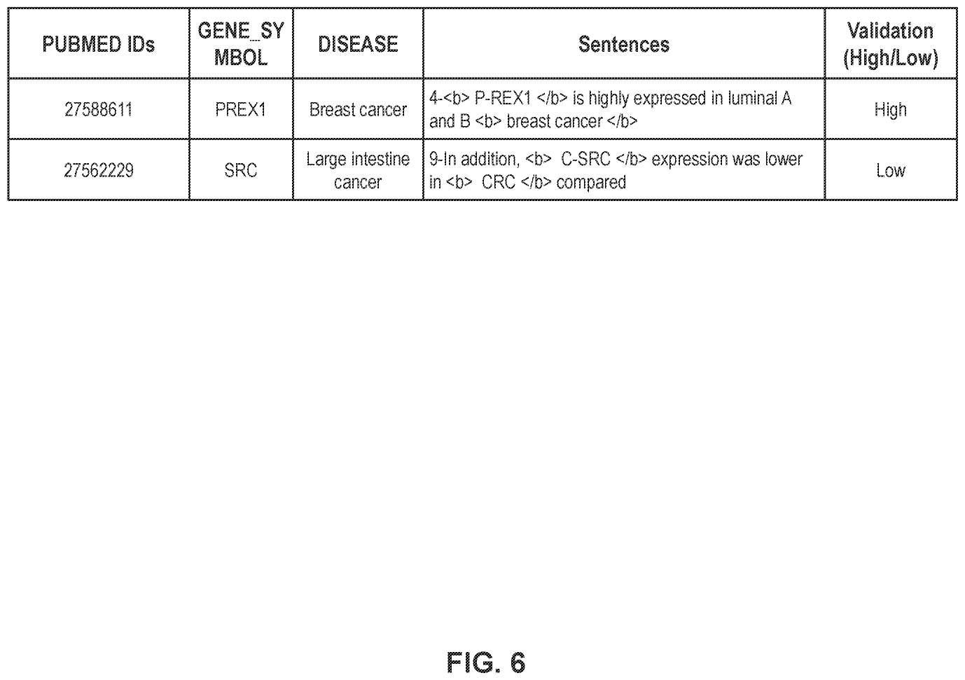

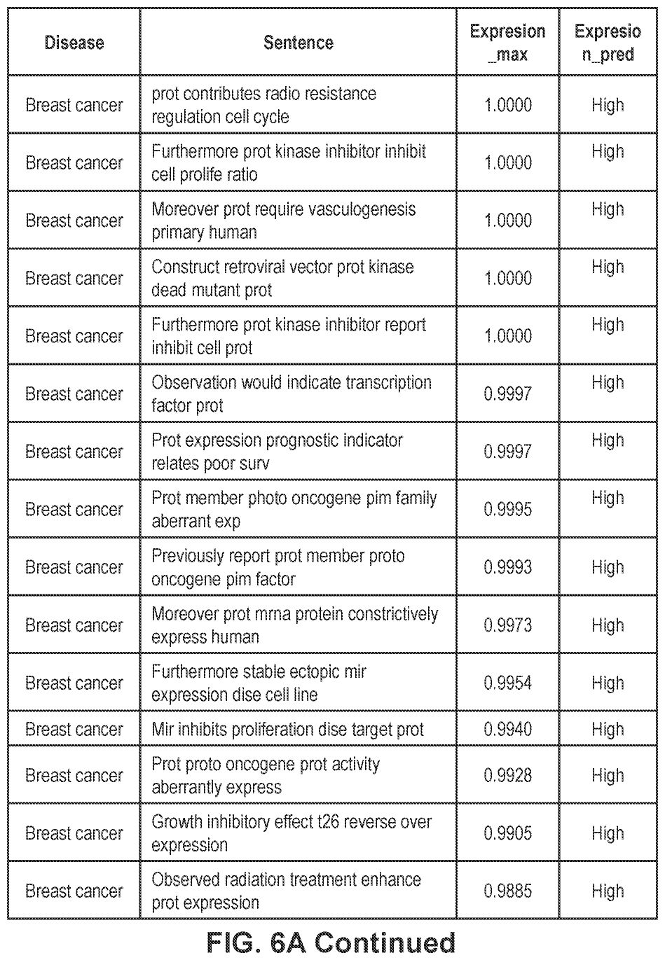

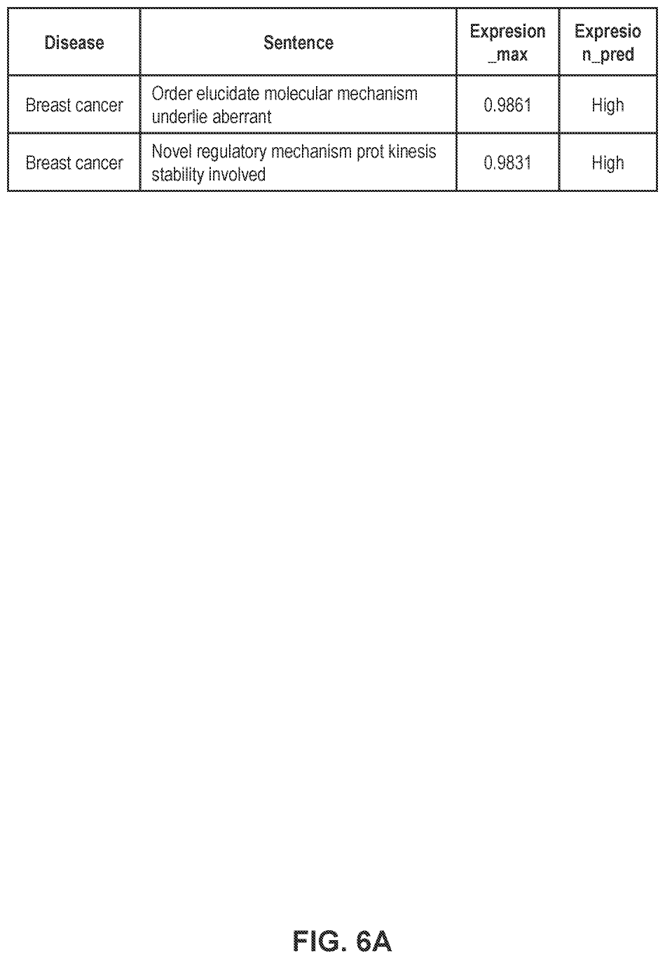

Further, as an example, FIG. 6 is a table illustrating a sample result set from the second-level logistic regression ML Classifier (124), in accordance with an embodiment of the invention. The second-level logistic regression ML Classifier (122) is configured for classifying based upon the expression level of the protein. Expression of a protein in disease condition is important to determine the curable action the drug needs to perform on the protein. This differential expression is traditionally calculated using a sample of healthy and disease data and large processing and time is needed for a single disease. Results of these experiments and computations are published as a part of the public database such as in biomedical literature and the like. Two labels are used: High and Low.

As is evident from the table in FIG. 6, out of 35 sentences (strings of words) validated for breast cancer for a protein, thirty showed HIGH expression and five showed Low expression.

In an embodiment, the second and third-level logistic regression ML Classifier (124 and 126) are each configured to assign a probability value to the each of the conclusive word string during classification.

As an example, FIG. 6A is a table illustrating a sample result set, wherein the strings of words are arranged in descending order of the probability as assigned by the second-level logistic regression ML Classifier (124), in accordance with an embodiment of the invention.

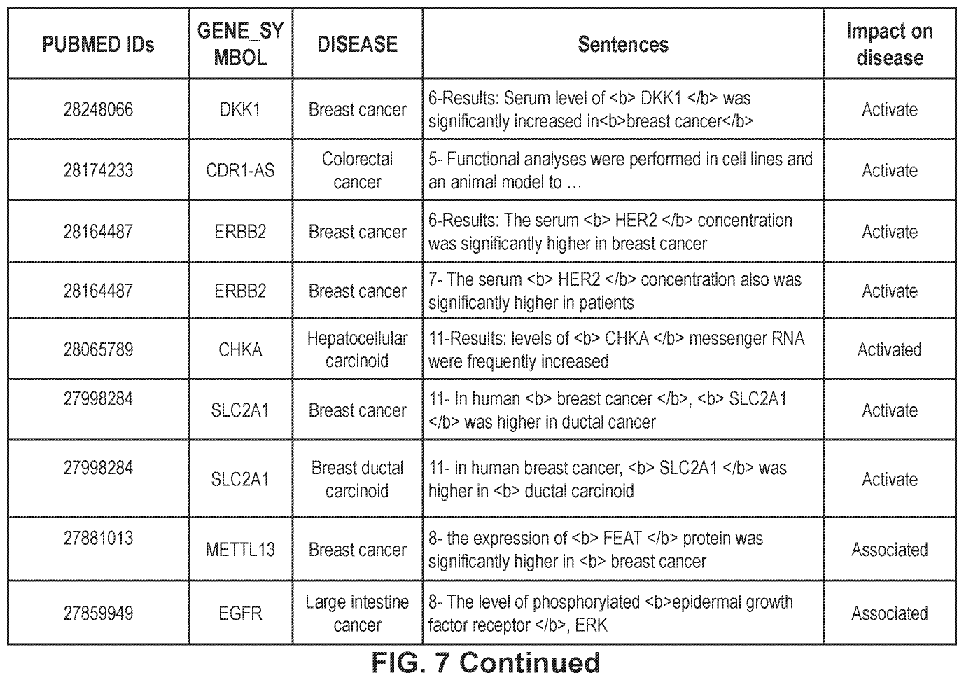

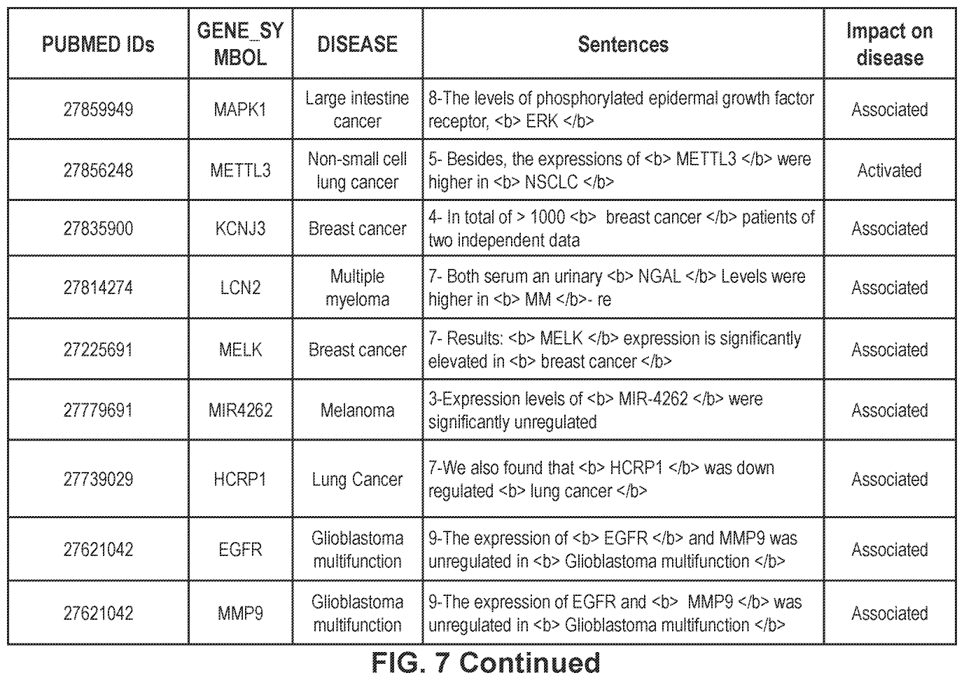

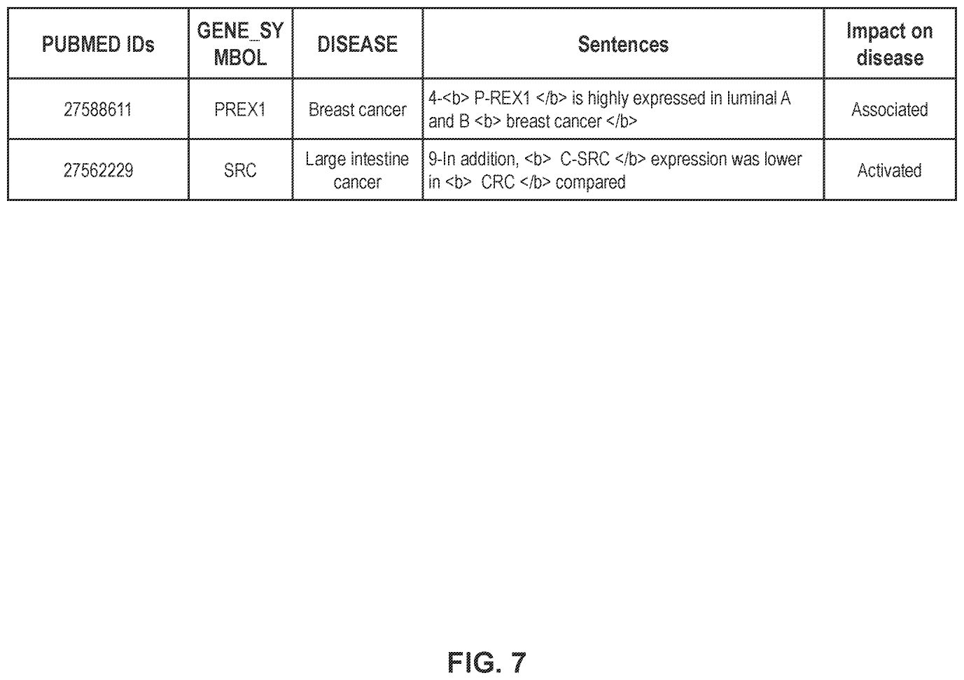

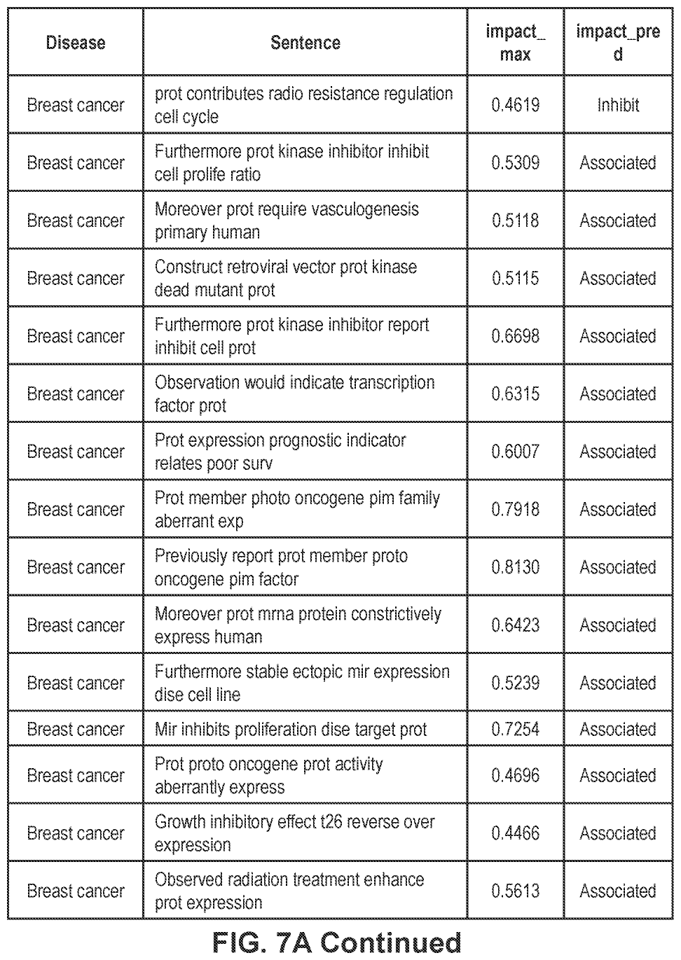

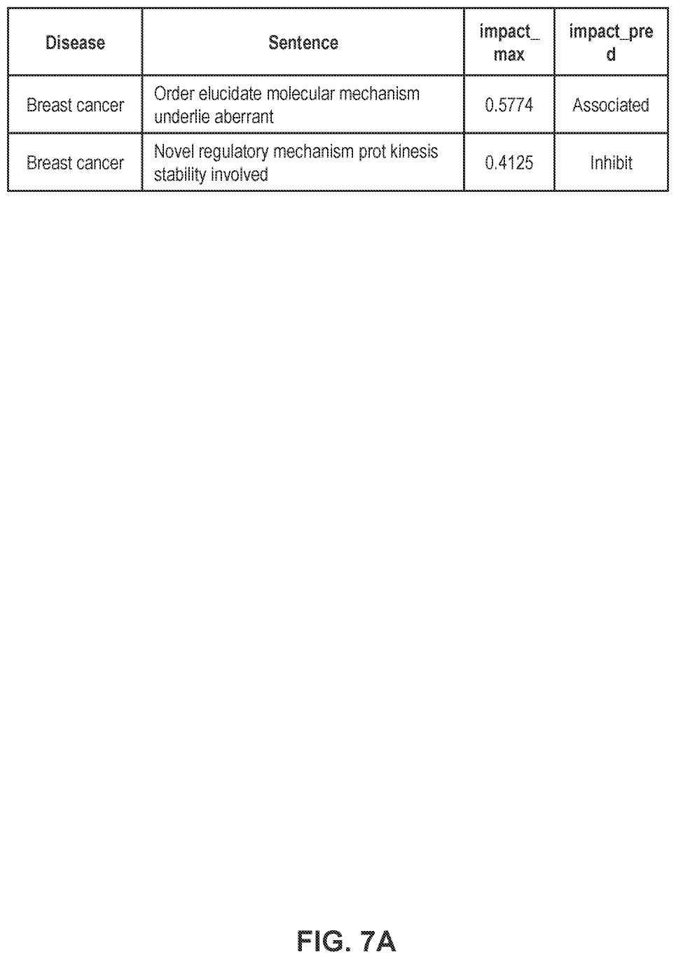

Further, as an example, FIG. 7 is a table illustrating a sample result set from the third-level logistic regression ML Classifier (126), in accordance with an embodiment of the invention. The third-level logistic regression ML Classifier (126) is configured for classifying based upon the impact of the protein on the disease. The effect of the protein expression level on the disease condition and the effect of that level on the disease are important to determine the therapeutic effect and determine the curable action of the target. The third-level logistic regression ML Classifier (126) is configured for classifying based upon this impact into three categories Inhibit, Associate, and Activate.

With reference to FIG. 7, the following cases can be seen as an example:

Case 1: If the protein suppresses the growth of the disease/indication, it is labelled as ‘Inhibit.’ For example: GPRC5A expression is frequently suppressed in majority of non-small cell lung cancers (NSCLCs). However, elevated GPRC5A is still observed in a small portion of NSCLC cell lines and tumors, suggesting that the tumor suppressive function of GPRC5A is inhibited in these tumors by an unknown mechanism.

Case 2: If there are no details regarding the role of protein in disease, it is labelled as ‘Associate.’ For example: Based on the protein expression analysis of a total of 275 patient samples, claudin-3 (CLDN3) expression is significantly increased in ADC tissues and is associated with cancer progression, correlating significantly with the poor survival of ADC patients (p=0.041&0.029).

Case 3: If the sentence specifies the activation or worsening of disease condition due to the protein the sentence is labeled as ‘Activate’. For example: M3R activation stimulates colon cancer cell invasion via cross-talk with epidermal growth factor receptors (EGFR), post-EGFR activation of mitogen-activated protein kinase (MAPK) extracellular signal-related kinase 1/2 (ERK1/2), and induction of matrix metalloproteinase-1 (MMP1) expression.

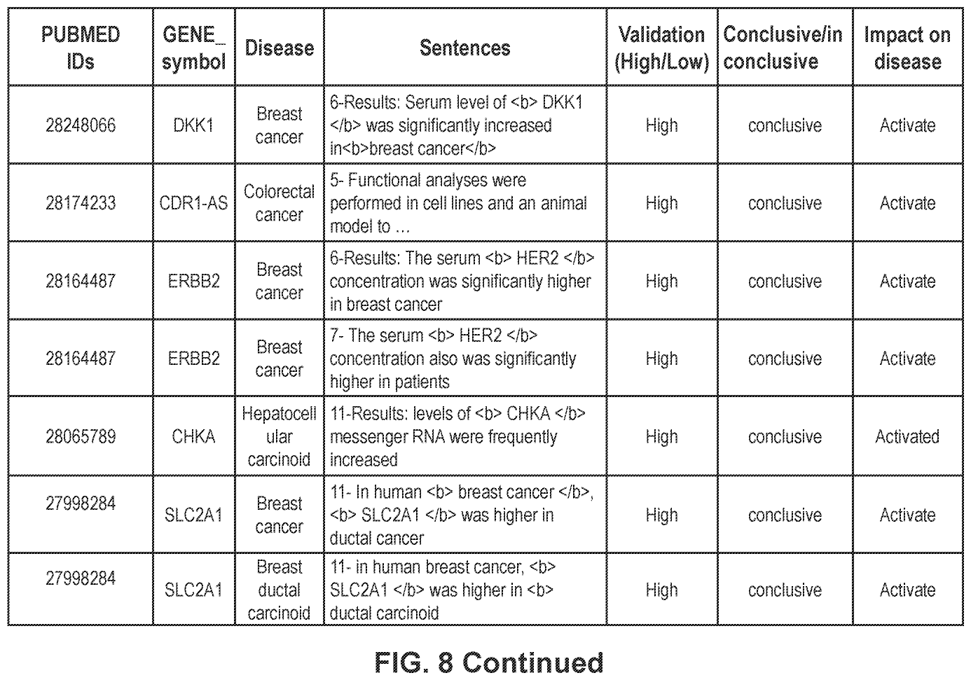

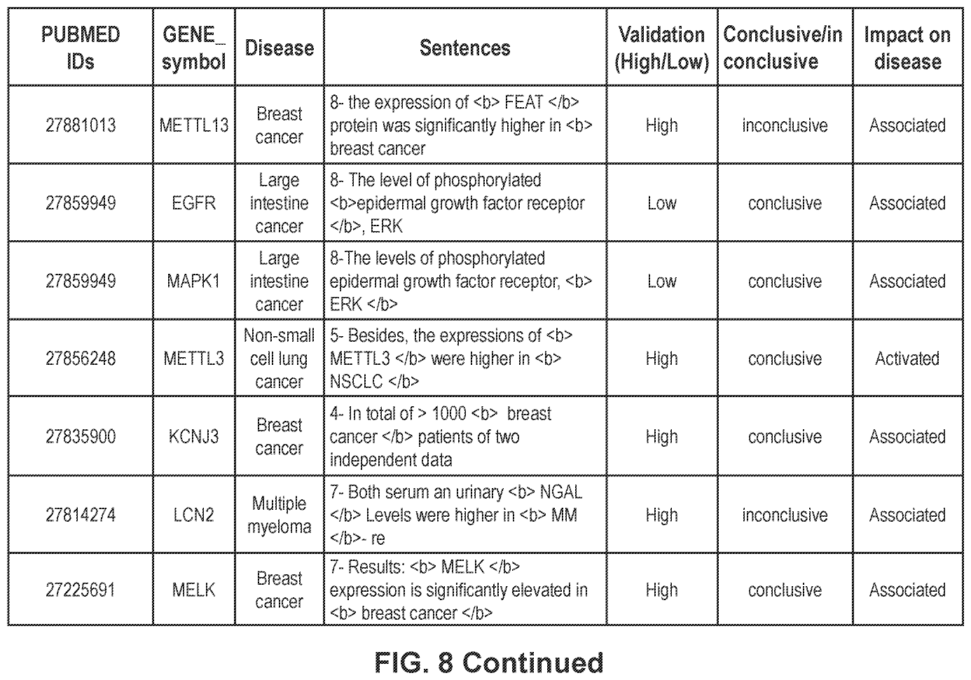

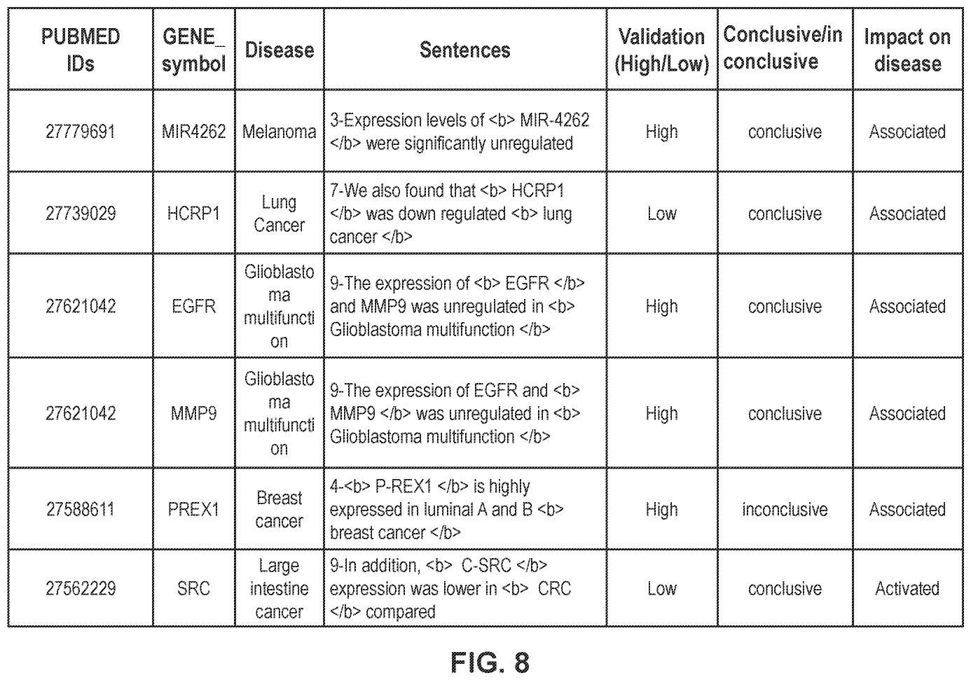

FIG. 8 is a table illustrating an aggregated result set incorporating the classification from all the three ML Classifiers (122, 124, 126), in accordance with an embodiment of the invention.

In an embodiment, the system (100) comprises a probability filtering module (130). The probability filtering module (130) is configured for identifying high probability word strings from the conclusive word strings by comparing an assigned probability value with a predefined threshold probability value. The predefined threshold probability value is chosen and input by a user of the system (100).

In an embodiment, the system (100) comprises a grouping module (140). The grouping module (140) is configured for aggregating the identified high probability word strings into groups based upon the disease to which the target protein in the high probability word strings pertains. The aggregated high probability word strings is the identified relevant information. The final expression level calculator (150) calculates the final expression level of the identified relevant information by comparing the probability values of the word strings.

With reference to the example embodiment, after the sentences get extracted and filtered through a bag of words, and the three classifiers (nature_of_sentence, expression_level, impact_on_disease), the corpus of sentences is narrowed down by a user defined threshold on the confidence probability using the probability filtering module (130) which is configured for identifying high probability word strings from the conclusive word strings by comparing the assigned probability value with a predefined threshold probability value. For example, a list of sentences may be represented as:

$$( S ) a [ protein ] + [ disease ] ,$$

$$where$$

$$S = s 1 , s 2 , s 3 … sn ]$$

FIG. 7A is a table illustrating a sample result set, wherein the strings of words are arranged with corresponding probability as assigned by the third-level logistic regression ML Classifier (126), in accordance with an embodiment of the invention.

The word strings/corpus of sentences are filtered based upon having a probability higher than the predefined threshold probability value, which may be defined by a user. The final expression level is calculated by the final expression level calculator (150) by subtracting the probability of high and low expression levels. This score is then used to predict the curable action of the target protein in the disease.

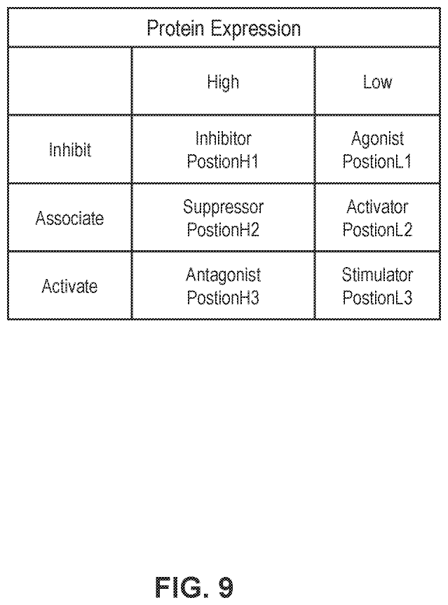

FIG. 9 is a table illustrating an example result of the system (100), in accordance with an embodiment of the invention. Based on the probability score of the protein, the target protein may be classified into one of the six positions in the matrix: PostionH1-3, PositionL1-3. Based upon this evaluation matrix, the curable action of a target protein in the related disease condition is predicted. This curable action with an assigned score aids in the process of drug development of the disease condition using the target protein.

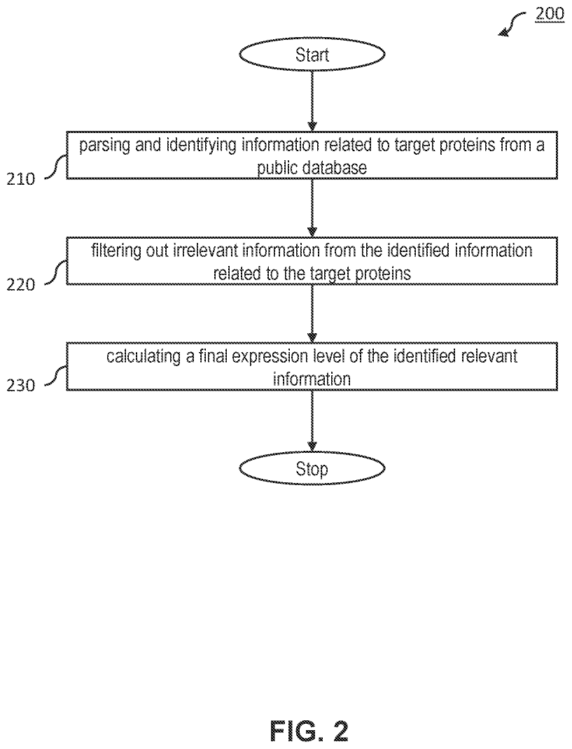

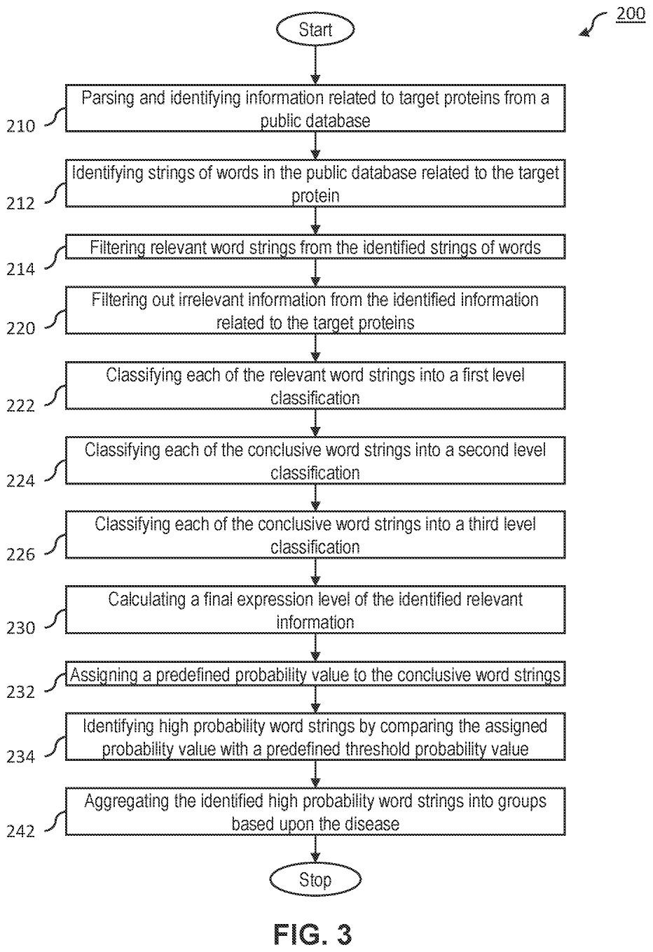

FIG. 2 is a flowchart illustrating a method (200) for determining a curable action value of a target protein for aiding drug development by determining a curable action value of a target protein, in accordance with an embodiment of the invention. The method (200) comprises the steps of parsing and identifying (210) information related to target proteins from a public database which comprises data related to proteins, filtering (220) out irrelevant information from the identified information related to the target proteins; calculating (230) a final expression level of the identified relevant information. The curable action of the target protein is based upon the final expression level.

In an embodiment, the step of parsing and identifying (210) comprises the steps of identifying (212) strings of words in the public database related to the target protein by searching for the target protein and the disease within the public database using a string extraction module (112); and filtering (214) relevant word strings from the identified strings of words by checking the presence of at least one word from a set of keywords (114) in the identified string of words.

In an embodiment, the step of filtering (220) comprises the steps of classifying (222) each of the relevant word strings into a first-level classification based upon the nature of the relevant word string into one of a conclusive word string and an inconclusive word string; classifying (224) each of the conclusive word strings into a second-level classification based upon the expression level of the protein into one of a high expression word string and a low expression word string; and classifying (226) each of the conclusive word strings into a third-level classification based upon the impact of the protein on the disease into one of an activate effect and an associate effect.

In an embodiment, the method (200) comprises the steps of assigning (232) a predefined probability value to the conclusive word strings; and identifying (234) high probability word strings from the conclusive word strings by comparing the assigned probability value with a predefined threshold probability value.

In an embodiment, the method (200) comprises the steps of aggregating (242) the identified high probability word strings into groups based upon the disease to which the target protein in the high probability word strings pertains. The aggregated high probability word strings form the identified relevant information. The step of calculating (230) includes comparing the probability values of the word strings.

In various embodiments, the steps of the method (200) may be performed using a Natural Language Processing (NLP) module (160).

Advantageously, the invention aids in determining the role of protein in a disease condition which indirectly would determine the Mechanism of Action (MoA) of the drug. MoA refers to the biochemical process through which a drug produces its effect and describes the functional changes at molecular or cellular level, and therefore, is a critical step in the drug discovery process. Determining the mechanism of action (MoA) of bioactive compounds is crucial as it helps in determination of better dose regimen, the clinical safety and best combination therapy that can reduce the likelihood of drug resistance.

The invention aids in drug development as it not only determines the role of protein in a disease but also predicts the curable action of a target protein based on the large biomedical literature corpus.

While the above detailed description has shown, described, and pointed out novel features as applied to various implementations, it can be understood that various omissions, substitutions, and changes in the form and details of the devices or algorithms illustrated can be made without departing from the scope of the disclosure. As can be recognized, certain implementations described herein can be embodied within a form that does not provide all of the features and benefits set forth herein, as some features can be used or practiced separately from others.

The foregoing description of the specific implementations will so fully reveal the general nature of the implementations herein that others can, by applying current knowledge, readily modify and/or adapt for various applications such specific implementations without departing from the generic concept, and, therefore, such adaptations and modifications should and are intended to be comprehended within the meaning and range of equivalents of the disclosed implementations. It is to be understood that the phraseology or terminology employed herein is for the purpose of description and not of limitation. Therefore, while the implementations herein have been described in terms of preferred implementations, those skilled in the art will recognize that the implementations herein can be practiced with modification within the spirit and scope of the invention as described herein.

We claim:

1. A system for determining a curable action value of a target protein for aiding in drug development, the system comprising:
a protein data extraction module, the protein data extraction module parsing and identifying information related to the target protein from a public database, the public database comprising data related to proteins;
a protein data filtration module for filtering out irrelevant information from the identified information related to the target proteins; and
a final expression level calculator, the final expression level calculator being configured for calculating the final expression level from the identified relevant information, the curable action of the target protein being based upon the final expression level.

2. The system according to claim 1, wherein the protein data extraction module comprises:
a string extraction module, the string extraction module identifying strings of words in the public database related to the target protein, the identification being done by searching for the target protein and a related disease within the public database;
a set of keywords, the set of keywords including a set of words; and
a string filtering module, the string filtering module filtering relevant word strings from the identified strings of words, the filtering being performed by checking the presence of at least one word from the set of keywords in the identified string of words.

3. The system according to claim 1, wherein the protein data filtration module comprises:
a first-level logistic regression ML Classifier, the first-level ML classifier classifying each of the relevant word strings into a first-level classification, the first level classification being one of a conclusive word string and an inconclusive word string, the first-level classification being done based upon the nature of the relevant word string;
a second-level logistic regression ML Classifier, the second-level ML classifier classifying each of the conclusive word strings into one of a high expression word string and a low expression word string, the second-level classification being done based upon the expression level of the protein; and
a third-level logistic regression ML Classifier, the third-level ML classifier classifying each of the conclusive word strings into one of an inhibit effect, an activate effect and an associate effect, the third-level classification being done based upon the impact of the protein on the related disease.

4. The system according to claim 3, wherein the set of keywords consists of at least one word chosen from a group consisting of mutat, express, polymorph, regulat, level, associat, inhibit, activat, high, low, less, up, and down.

5. The system according to claim 1, wherein the system comprises probability filtering module, the probability filtering module configured to identify high probability word strings from the conclusive word strings by comparing an assigned probability value with a predefined threshold probability value, the predefined threshold probability value being input by a user of the system.

6. The system according to claim 5, wherein the system comprises a grouping module, the grouping module being configured to aggregate the identified high probability word strings into groups based upon the disease to which the target protein in the high probability word strings pertains, the aggregated high probability word strings being the identified relevant information.

7. The system according to claim 1, wherein the final expression level calculator calculates the final expression level from the identified relevant information by comparing the probability values of the word strings.

8. The system according to claim 1, wherein the public data base is a dynamic digital database.

9. The system according to claim 1, wherein the system includes a Natural Language Processing (NLP) module, wherein the protein data extraction module uses the NLP module for parsing and identifying information related to the target proteins.

10. A method for determining a curable action value of a target protein for aiding in drug development by, the method comprising the steps of:
parsing and identifying information related to the target protein from a public database, the public database comprising data related to proteins;
filtering out irrelevant information from the identified information related to the target proteins; and
calculating a final expression level of the identified relevant information, the curable action of the target protein being based upon the final expression level.

11. The method according to claim 10, wherein the step of parsing and identifying comprises the steps of:
identifying strings of words in the public database related to the target protein, the identifying being done by searching for the target protein and a related disease within the public database using a string extraction module; and
filtering relevant word strings from the identified strings of words, the filtering being performed by checking the presence of at least one word from a set of keywords in the identified string of words using a string filtering module.

12. The method according to claim 10, wherein the step of filtering comprises the steps of:
classifying each of the relevant word strings into a first-level classification using a first-level logistic regression ML Classifier, the classifying being done based upon the nature of the relevant word string into one of a conclusive word string and an inconclusive word string;
classifying each of the conclusive word strings into a second-level classification using a second-level logistic regression ML Classifier, the classifying being done based upon the expression level of the protein into one of a high expression word string and a low expression word string; and
classifying each of the conclusive word strings into a third-level classification using a third-level logistic regression ML Classifier, the classifying being done based upon the impact of the protein on the related disease into one of an activate effect and an associate effect.

13. The method according to claim 10, wherein the method comprises the steps of identifying high probability word strings from the conclusive word strings by comparing an assigned probability value with a predefined threshold probability value, the predefined threshold probability value being input by a user of the system.

14. The method according to claim 13, wherein the method comprises the steps of aggregating, using a grouping module, the identified high probability word strings into groups based upon the disease to which the target protein in the high probability word strings pertains, the aggregated high probability word strings being the identified relevant information.

15. The method according to claim 10, wherein the step of calculating includes calculating the final expression level from the identified relevant information by comparing the probability values of the word strings. The method according to claim 10, wherein the steps of the method are performed using a Natural Language Processing (NLP) module.
